(19) United States
(12) Patent Application Publication
(10) Pub. No.: US 2026/0020774 A1
(43) Pub. Date: Jan. 22, 2026

(54) APPARATUS AND METHOD FOR NON-INVASIVELY MONITORING ARTERIAL BLOOD CARBON DIOXIDE DURING SURGERY

(71) Applicants: JEONBUK NATIONAL UNIVERSITY HOSPITAL, Jeonbuk-do (KR); INDUSTRIAL COOPERATION FOUNDATION JEONBUK NATIONAL UNIVERSITY, Jeonbuk-do (KR); SEOUL NATIONAL UNIVERSITY HOSPITAL, Seoul (KR)

(72) Inventors: HYUN HO KIM, Jeonbuk-do (KR); AH RA LEE, Gyeonggi-do (KR)

(21) Appl. No.: 19/270,663

(22) Filed: Jul. 16, 2025

(30) Foreign Application Priority Data
Jul. 18, 2024 (KR) 10-2024-0095080

(51) Int. Cl. A61B 5/08; A61B 5/00; A61B 5/01; A61B 5/1455; G16H 10/60; G16H 50/30; G16H 50/70

(52) U.S. Cl. CPC A61B 5/08; A61B 5/01; A61B 5/14551; A61B 5/7275; G16H 10/60; G16H 50/30; G16H 50/70

(57) ABSTRACT

A method for non-invasively monitoring arterial blood carbon dioxide during surgery include collecting and storing biometric signal information during surgery of patients, clinical information before the surgery, and end-tidal carbon dioxide (ETCO₂) and partial pressure of arterial carbon dioxide (PaCO₂), generating learning data in which the biometric signal information during the surgery, the clinical information before the surgery, and the ETCO₂ are input conditions and the PaCO₂ is an output condition on the basis of a data collection result and then allowing a prediction model to perform machine learning on a correlation between the ETCO₂ and the PaCO₂, acquiring and storing clinical information before surgery of the surgical patient, and, when the surgery of the surgical patient is started, predicting PaCO₂ in real time by measuring biometric signal information and ETCO₂ and then analyzing the biometric signal information and the ETCO₂ before the surgery through the prediction model.

### CROSS-REFERENCE TO RELATED APPLICATION AND CLAIM OF PRIORITY

This application claims the benefit under 35 USC § 119 of Korean Patent Application No. 10-2024-0095080 filed on Jul. 18, 2024, in the Korean Intellectual Property Office, the entire disclosure of which is incorporated herein by reference for all purposes.

### BACKGROUND

### 1 Technical Field

The present disclosure relates to an apparatus and method for non-invasively monitoring arterial blood carbon dioxide during surgery, which enables the non-invasive, real-time monitoring of arterial blood carbon dioxide during a patient's surgery.

### 2 Background Art

The partial pressure of arterial carbon dioxide (PaCO₂) and end-tidal carbon dioxide (ETCO₂) are important indicators of respiratory function and are used as standard monitoring elements in general anesthesia surgery.

Recent research has reported that differences between PaCO₂ and ETCO₂ may be used to evaluate and predict a patient's state in various environments from transfer to a hospital to treatment during surgery, and have demonstrated that a difference between PaCO₂ and ETCO₂ may influence a patient's death rate when the difference between PaCO₂ and ETCO₂ is 10 mmHg or more.

In addition, it has been discovered that a correlation exists between the death rate of a patient who undergoes trauma surgery or a patient who may suffer from septicemia and the difference between PaCO₂ and ETCO₂ of the patient. This discovery highlights the potential of the difference between PaCO₂ and ETCO₂ as an attractive means of assisting in patient management and guiding resuscitation.

However, while ETCO₂ can be measured using a non-invasive method, PaCO₂ can only be obtained through an invasive technique known as arterial blood gas analysis (ABGA). Hence, there is a problem that it is difficult to continuously measure PaCO₂ during surgery performed in a state where a patient is put under general anesthesia.

In particular, there may occur additional problems that it is inconvenient for an anesthesiologist participating in surgery to continuously monitor arterial pressure during the surgery and measure arterial blood carbon dioxide through an ABGA test by collecting arterial blood and that there occurs a phenomenon that measurement noise is generated for a few seconds during blood collection and blood pressure suddenly rises as a measured arterial blood flow rate changes.

### SUMMARY

The present disclosure is designed to address the above-described problems. Therefore, the present disclosure is directed to providing an apparatus and method for non-invasively monitoring arterial blood carbon dioxide during surgery, which enables real-time monitoring of arterial blood carbon dioxide even during surgery, utilizing an artificial intelligence method.

The present disclosure is further directed to providing an apparatus and a method for non-invasively monitoring arterial blood carbon dioxide during surgery. This enables the detection of patient features by reflecting both biometric signal information during surgery and pre-surgery information, and facilitates a more precise prediction operation by incorporating the detected features.

Objectives of the present disclosure are not limited to the above-described objectives, and other objectives of the present disclosure, which are not mentioned, will be apparent and understood by those skilled in the art from the following description.

To achieve the above-described objective, a method for non-invasively monitoring arterial blood carbon dioxide during surgery, in which an apparatus for non-invasively monitoring arterial blood carbon dioxide predicts end-tidal carbon dioxide-based arterial blood carbon dioxide during the surgery, includes a data collection step of collecting and storing biometric signal information during surgery of a plurality of patients, clinical information before the surgery, and end-tidal carbon dioxide (ETCO₂) and partial pressure of arterial carbon dioxide (PaCO₂); a prediction model learning step of generating a plurality of learning data in which the biometric signal information during surgery, the clinical information before the surgery, and the ETCO₂ are input conditions and the PaCO₂ is an output condition on the basis of a data collection result and then allowing a prediction model to perform machine learning on a correlation between the ETCO₂ and the PaCO₂ through the learning data; a surgical patient determination step of, when a surgical patient is determined, acquiring and storing clinical information before surgery of the surgical patient; and a prediction step of, when the surgery of the surgical patient is started, predicting PaCO₂ in real time by measuring biometric signal information during surgery and ETCO₂ and then analyzing the biometric signal information during the surgery and the ETCO₂ along with the clinical information before the surgery through the prediction model.

Preferably, the biometric signal information collected during surgery includes body temperature, inspired oxygen fraction, percutaneous oxygen saturation, airway compliance, and tidal volume index.

Preferably, the clinical information provided before surgery includes at least one of the following: age, gender, weight, body mass index, type of surgery, surgical method, surgical site, creatinine level, albumin level, hemoglobin level, and pulmonary function test results.

Preferably, the prediction model is implemented using one of the following: random forest, logistic regression, or XGBoost.

Preferably, the method further includes a monitoring step of calculating and notifying the patient of the degree of risk based on the PaCO₂ or the difference between the ETCO₂ and the PaCO₂.

To achieve the above-described objective, an apparatus for non-invasively monitoring arterial blood carbon dioxide during surgery, the apparatus includes a prediction model implemented through any one of random forest, logistic regression, and XGBoost; a data collection unit configured to collect and store biometric signal information during surgery of a plurality of patients, clinical information before the surgery, and end-tidal carbon dioxide (ETCO₂) and partial pressure of arterial carbon dioxide (PaCO₂); a prediction model learning unit configured to generate a plurality of learning data in which the biometric signal information during the surgery, the clinical information before the surgery, and the ETCO₂ are input conditions and the PaCO₂ is an output condition on the basis of a data collection result and then allow the prediction model to perform machine learning on a correlation between the ETCO₂ and the PaCO₂ through the learning data; and a prediction unit configured to, when a surgical patient is determined, acquire and store clinical information before surgery of the surgical patient, and when the surgery of the surgical patient is started, predict and output PaCO₂ in real time by measuring biometric signal information during the surgery and ETCO₂ and then analyzing the biometric signal information during the surgery and the ETCO₂ along with the clinical information before the surgery through the prediction model, wherein the biometric signal information during the surgery includes a body temperature, an inspired oxygen fraction, a percutaneous oxygen saturation, an airway compliance, and a tidal volume index, and wherein the clinical information before the surgery includes at least one of an age, a gender, a weight, a body mass index, a surgical type, a surgical method, a surgical site, a creatinine level, an albumin level, a hemoglobin level, and a pulmonary function test result.

In the present disclosure, arterial blood carbon dioxide is non-invasively predicted based on end-tidal carbon dioxide using an artificial intelligence method, allowing for real-time monitoring of arterial blood carbon dioxide even during surgery.

Furthermore, patient features are detected by reflecting clinical information from both during and before surgery, and arterial blood carbon dioxide is predicted by reflecting these detected features, allowing for an optimized prediction result to be provided to each patient.

### BRIEF DESCRIPTION OF THE DRAWINGS

FIG. 1 illustrates a method for non-invasively monitoring arterial blood carbon dioxide during surgery, according to an embodiment of the present disclosure.

FIG. 2 is a diagram showing the implementation of a prediction model according to an embodiment of the present disclosure.

FIG. 3 is a diagram illustrating an apparatus for non-invasively monitoring arterial blood carbon dioxide during surgery according to an embodiment of the present disclosure.

### DETAILED DESCIPTION

Hereinafter, preferred embodiments will be described in detail such that those of ordinary skill in the art can easily practice the present disclosure regarding the accompanying drawings. However, in describing a preferred embodiment of the present disclosure in detail, if it is determined that a detailed description of a related known function or configuration unnecessarily obscures the gist of the present disclosure, the detailed description thereof will be omitted. Additionally, the same reference numerals may be used throughout the drawings for parts that have similar functions and operations.

Throughout the entire specification, when an element is referred to as being “connected” or “coupled” to another element, it can be directly connected or coupled to the other element, or it can be indirectly connected or coupled to the other element with one or more intervening elements interposed between them. In addition, it will be understood that when a component “includes” an element, unless there is another opposite description thereto, it should be understood that the component does not exclude another element but may further include another element.

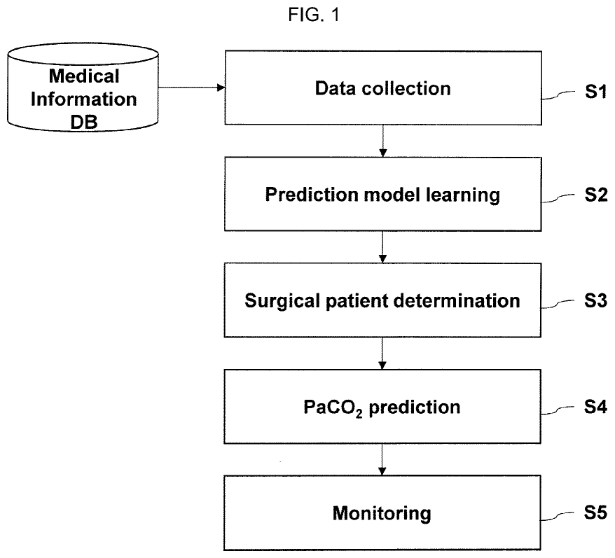

FIG. 1 illustrates a method for non-invasively monitoring arterial blood carbon dioxide during surgery, according to an embodiment of the present disclosure.

Referring to FIG. 1, the method of the present disclosure includes a data collection step S1, a prediction model learning step S2, a surgical patient determination step S3, a PaCO₂ prediction step S4, a monitoring step S5, and the like.

### Data Collection Step S1

Among patients for whom surgery has been completed, those with respiratory abnormalities are selected at a fixed ratio, along with normal patients.

In addition, a plurality of data sets are generated and stored by extracting biometric signal information during surgery of each selected patient, clinical information before the surgery, ETCO₂, and PaCO₂, using a medical information database in which all information related to patients is collected and stored, and editing this information according to a preset data rule.

The medical information database may be a database managed by a specific medical center, a cloud server, or the like, such as Vital DB. The patient is preferably 18 years old or older, undergoes general anesthesia, has an American Society of Anesthesiologists Physical Status Classification (ASA-PS) class V or less, and allows for PaCO₂ measurement once or more. However, the present disclosure is not limited thereto.

The biometric signal information collected during surgery may include body temperature, inspired oxygen fraction, percutaneous oxygen saturation, airway compliance, and tidal volume index. It may additionally include at least one of a blood pressure, a capnography reference respiration rate, a positive end-expiratory pressure, a plateau pressure, a tidal oxygen fraction, leakage of a respirator, a minimal alveolar concentration of a volatile substance, and an average tidal volume per kg of normal lean body weight, when necessary.

The clinical information provided before surgery may include at least one of the following: age, gender, weight, body mass index, type of surgery, surgical method, surgical site, creatinine level, albumin level, hemoglobin level, and pulmonary function test results.

The ETCO₂ and the PaCO₂ are preferably acquired and stored through time synchronization. However, there is a problem that it is impossible to determine the exact time of an ABGA test for measuring PaCO₂, and it is only possible to check the time at which the ABGA test result is input into the database.

For reference, a phenomenon may occur where, when an anesthesiologist continuously monitors arterial pressure through an arterial line during surgery and collects arterial blood, measurement noise is generated for a few seconds during the blood collection process. As a result, blood pressure may suddenly rise due to changes in the measured arterial blood flow rate. Therefore, in the present disclosure, the latest observation time at which an average blood pressure within 20 minutes before PaCO₂ was recorded has suddenly risen abnormally is assumed as an ABGA test time, and PaCO₂ and ETCO₂, which are acquired at the ABGA test time, are determined as information acquired at a same time.

### Prediction Model Learning Step S2

A plurality of learning data in which the biometric signal information during the surgery, the clinical information before the surgery, and the ETCO₂ are input conditions and the PaCO₂ is an output condition are generated based on the plurality of data sets, and then divided into training and verification sets at a ratio of 8:2 according to a numerical distribution.

After descriptive statistical analysis is performed on both the training and verification sets, categorical variables are converted using a one-hot encoding method, and continuous variables are converted using a minimum-maximum scaling method.

Each learning dataset is preprocessed to serve as an input variable for a machine learning model, and missing values are removed so that all prediction variables have missing values of less than 10% in both the training and verification sets. Additionally, outliers are removed based on the results of previous documents.

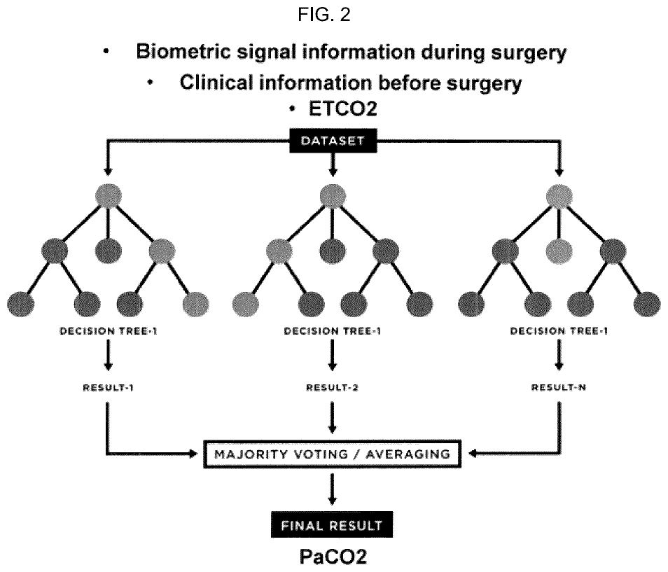

In addition, as shown in FIG. 2, a prediction model is implemented using a random forest that applies ensemble learning, specifically, bootstrap aggregation (bagging).

Alternatively, prediction models may be implemented using logistic regression, Extreme Gradient Boost (XGBoost), and similar methods, instead of the random forest; however, the present disclosure is not necessarily limited thereto.

In addition, an operation of allowing the prediction model to perform machine learning on a correlation between the ETCO₂ and the PaCO₂ through the training sets and verifying prediction performance of the prediction model through the verification sets is repeated, and the machine learning is finished when the prediction accuracy of the prediction model satisfies a preset target value.

### Surgical Patient Determination Step S3

When a surgical patient is determined to be in a state where the machine learning of the prediction model is complete, clinical information about the patient is acquired and stored in advance by accessing a database of the medical center where the surgery is to be performed.

### PaCO₂ Prediction Step S4

When the surgery of a surgical patient is started, biometric signal information during the surgery and ETCO₂ are repeatedly measured using various medical devices installed in the operating room.

In addition, whenever clinical information and ETCO₂ are newly measured during surgery, the new measurement information, along with pre-acquired clinical information from before the surgery, is input into the prediction model, which then predicts the current PaCO₂.

### Monitoring Step S5

Finally, the currently predicted PaCO₂ is displayed in real-time on the screen of a monitoring device.

Additionally, at least one of a PaCO₂ abnormality detection reference and an upper limit of differences between ETCO₂ and PaCO₂ is predefined, and a degree of risk is preset accordingly. When the currently predicted PaCO₂ value satisfies the PaCO₂ abnormality detection reference, alarm information is generated and output to enable the medical team to detect the alarm and take follow-up measures.

Alternatively, an upper limit for the difference between ETCO₂ and PaCO₂ is preset. Even when the difference between the currently predicted PaCO₂ and the presently measured ETCO₂ exceeds this preset upper limit, alarm information is generated and output, enabling the medical team to take immediate follow-up measures to address the current critical situation.

As such, in the present disclosure, biometric signal information obtained during surgery, clinical information obtained before surgery, and ETCO₂, which can be non-invasively measured during surgery, are analyzed using an artificial intelligence method, thereby enabling non-invasive measurement of PaCO₂.

Furthermore, PaCO₂ can be predicted by assessing a patient's state based on biometric signal information gathered during surgery and before surgery, thereby reflecting the patient's actual state. This allows for an optimized prediction operation tailored to each patient's specific state.

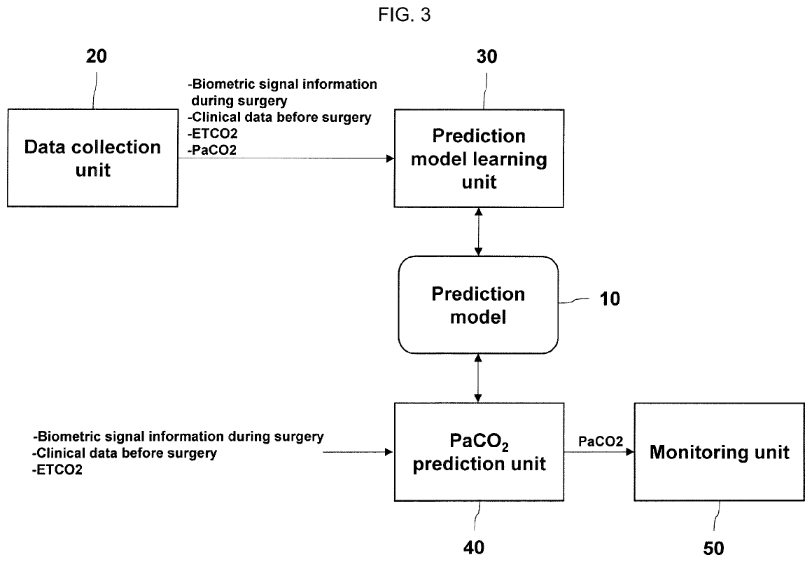

FIG. 3 is a diagram illustrating an apparatus for non-invasively monitoring arterial blood carbon dioxide during surgery according to an embodiment of the present disclosure.

Referring to FIG. 3, the apparatus of the present disclosure includes a prediction model 10 implemented through any one of random forest, logistic regression, and XGBoost; a data collection unit 20 which collects and stores biometric signal information during surgery of a plurality of patients, clinical information before the surgery, and end-tidal carbon dioxide (ETCO₂) and partial pressure of arterial carbon dioxide (PaCO₂); a prediction model learning unit 30 which generates a plurality of learning data in which the biometric signal information during surgery, the clinical information before the surgery, and the ETCO₂ are input conditions and the PaCO₂ is an output condition on the basis of a data collection result and then allows the prediction model to perform machine learning on a correlation between the ETCO₂ and the PaCO₂ through the learning data; a prediction unit 40 which, when a surgical patient is determined, acquires and stores clinical information before surgery of the surgical patient, and when the surgery of the surgical patient is started, predicts and outputs PaCO₂ in real time by measuring biometric signal information during the surgery and ETCO₂ and then analyzing the biometric signal information during surgery and the ETCO₂ along with the clinical information before the surgery through the prediction model; a monitoring unit 50 which calculates and notifies a degree of risk of the patient on the basis of whether or not the PaCO₂ satisfies an abnormality detection reference or a difference between the ETCO₂ and the PaCO₂; and the like.

Although limited embodiments and drawings have been described in the present disclosure, it is obvious that the present disclosure is not limited thereto. Still, various modifications and variations are possible by those of ordinary skill in the technical field to which the present disclosure belongs. Therefore, the spirit of the present disclosure should not be limited to the embodiments described. Still, the claims and all modifications equal or equivalent to the claims are intended to fall within the spirit of the present disclosure.

Additionally, the apparatus and method according to the present disclosure may be implemented as computer-readable code in computer-readable recording media. The computer-readable recording media include all kinds of recording devices in which data readable by a computer system is stored. Examples of the computer-readable recording media include ROM, RAM, CD-ROM, magnetic tape, floppy disk, optical data storage device, hard disk, flash drive, and the like, and may also be realized in the form of a carrier wave (for example, transmission through the Internet). In addition, the computer-readable recording media may be distributed to a computer system connected through networks to store and implement the computer-readable codes using a distribution mechanism.

What is claimed is:

1. A method for non-invasively monitoring arterial blood carbon dioxide during surgery, in which an apparatus for non-invasively monitoring arterial blood carbon dioxide predicts end-tidal carbon dioxide-based arterial blood carbon dioxide during the surgery, the method comprises:
a data collection step of collecting and storing biometric signal information during surgery of a plurality of patients, clinical information before the surgery, and end-tidal carbon dioxide (ETCO₂) and partial pressure of arterial carbon dioxide (PaCO₂);
a prediction model learning step of generating a plurality of learning data in which the biometric signal information during the surgery, the clinical information before the surgery, and the ETCO₂ are input conditions and the PaCO₂ is an output condition based on a data collection result and then allowing a prediction model to perform machine learning on a correlation between the ETCO₂ and the PaCO₂ through the learning data;
a surgical patient determination step of, when a surgical patient is determined, acquiring and storing clinical information before surgery of the surgical patient; and
a prediction step of, when the surgery of the surgical patient is started, predicting PaCO₂ in real-time by measuring biometric signal information during surgery, ETCO₂, and then analyzing the biometric signal information during the surgery along with the ETCO₂, as well as the clinical information before the surgery, through the prediction model.

2. The method for non-invasively monitoring arterial blood carbon dioxide during surgery according to claim 1,
wherein the biometric signal information during the surgery includes body temperature, inspired oxygen fraction, percutaneous oxygen saturation, airway compliance, and tidal volume index.

3. The method for non-invasively monitoring arterial blood carbon dioxide during surgery according to claim 1,
wherein the clinical information before surgery includes at least one of the following: age, gender, weight, body mass index, surgical type, surgical method, surgical site, creatinine level, albumin level, hemoglobin level, and pulmonary function test result.

4. The method for non-invasively monitoring arterial blood carbon dioxide during surgery according to claim 1,
wherein the prediction model is implemented using any one of the following: random forest, logistic regression, and XGBoost.

5. The method for non-invasively monitoring arterial blood carbon dioxide during surgery according to claim 1, further comprising:
a monitoring step of calculating and notifying the degree of risk of the patient based on the PaCO₂ or a difference between the ETCO2 and the PaCO₂.

6. An apparatus for non-invasively monitoring arterial blood carbon dioxide during surgery, the apparatus comprising:
a prediction model implemented through any one of random forest, logistic regression, and XGBoost;
a data collection unit configured to collect and store biometric signal information during surgery of a plurality of patients, clinical information before the surgery, and end-tidal carbon dioxide (ETCO₂) and partial pressure of arterial carbon dioxide (PaCO₂);
a prediction model learning unit configured to generate a plurality of learning data in which the biometric signal information during the surgery, the clinical information before the surgery, and the ETCO₂ are input conditions and the PaCO₂ is an output condition based on a data collection result and then allow the prediction model to perform machine learning on a correlation between the ETCO₂ and the PaCO₂ through the learning data; and
a prediction unit configured to, when a surgical patient is determined, acquire and store clinical information before surgery of the surgical patient, and when the surgery of the surgical patient is started, predict and output PaCO₂ in real time by measuring biometric signal information during the surgery and ETCO₂ and then analyzing the biometric signal information during the surgery and the ETCO₂ along with the clinical information before the surgery through the prediction model,
wherein the biometric signal information during surgery includes a body temperature, an inspired oxygen fraction, a percutaneous oxygen saturation, an airway compliance, and a tidal volume index, and
wherein the clinical information before surgery includes at least one of the following: age, gender, weight, body mass index, surgical type, surgical method, surgical site, creatinine level, albumin level, hemoglobin level, and pulmonary function test result.
